(19) United States
(12) Patent Application Publication
(10) Pub. No.: US 2018/0320218 A1
(43) Pub. Date: Nov. 8, 2018

(54) KIT FOR POLYMERASE CHAIN REACTION

(71) Applicant: SMOBiO Technology, Inc., Hsinchu City (TW)

(72) Inventors: CHUN-HSIEN KUO, Hsinchu City (TW); KUAN-LIN LEE, Hsinchu City (TW); CHEN-SHENG WU, Hsinchu City (TW); YI-YUN WANG, Hsinchu City (TW)

(73) Assignee: SMOBiO Technology, Inc., Hsinchu City (TW)

(21) Appl. No.: 15/589,368

(22) Filed: May 8, 2017

(51) Int. Cl. C12Q 1/68

(52) U.S. Cl. CPC C12Q 1/686; C12Q 1/6848

(57) ABSTRACT

The invention provides a kit for Polymerase Chain Reaction (PCR), comprising a vessel and at least two set of reagents disposed separately in the vessel, wherein each of the at least two set of reagents comprise at least one component selected from the group consisting of a DNA polymerase, nucleoside triphosphates, reaction buffer, and salt, and provided that each set of the at least two set of reagents is different from each other and combination of each of the at least two set of reagents comprises a DNA polymerase, nucleoside triphosphates, reaction buffer, and salt required for performing PCR.

### BACKGROUND OF THE INVENTION

### Field of the Invention

The present invention relates to a kit for polymerase chain reaction, more specifically, to a pre-mixed kit for polymerase chain reaction which is stable to store at room temperature or above.

### Description of Prior Art

DNA polymerase chain reaction (hereinafter referred to as “PCR”) allows the DNA sequence at a specific region of a genome to be amplified by more than a million-fold, provided that at least part of its nucleotide sequence is already known. Portions of the sequence that surround the region to be amplified are used to design two synthetic DNA oligonucleotides, one complementary to each strand of the DNA double helix. These oligonucleotides serve as primers for in vitro DNA synthesis, which is catalyzed by a DNA polymerase, and they determine the ends of the final DNA fragment that is obtained. Each cycle of the PCR requires denaturation to separate two strands of the DNA double helix, annealing for specific hybridization to complementary DNA sequences, and extension for synthesis of DNA. For effective amplification, 30 to 40 cycles of reaction are required.

In conclusion, the PCR by which a specific nucleotide sequence can be amplified in vitro from the genomic DNA, enables the detection of a specific DNA sequence and the acquisition of the DNA fragment of interest in a large quantity in a short period of time.

The PCR technique has been utilized in a wide range of life sciences, such as in the detection of genes associated with genetic diseases; in the detection and expression of a specific MRNA by way of cDNA amplification by employing reverse transcription-PCR (RT-PCR) and RACE (rapid amplification of cDNA end) methods; in direct nucleotide sequencing from the amplified product of DNA; in the analysis of VNTR (various number of tandem repeat); and, in genetic mapping. In addition, it has been also utilized in the diagnosis of a variety of diseases, such as HTLV-I (human T-cell lymphoma/leukemia virus type I), HIV (human immunodeficiency virus) and HBV (hepatitis B virus), and currently its usefulness becomes more and more widespread.

Furthermore, applications of said PCR technique, e.g., DD-PCR (differential display-PCR) and Immuno-PCR have been developed, which permit detection of only a small portion of RNA or DNA in question not detectable using currently available methods.

In the amplification of nucleic acid by PCR technique, every component of the reaction mixture for PCR, i.e., a template DNA, primers, reaction buffer, MgCl₂, KCl, dNTPs (dATP, dCTP, dGTP and dTTP) and DNA polymerase, must be mixed in step-wise fashion or simultaneously at the initial step, prior to initiating the reaction. Accordingly, it has been cumbersome to add and mix the trace amounts of each component in a separate manner for every test sample, so experimental errors have been frequently accompanied. Especially when numerous samples are to be analyzed in a short period of time, the inefficiency and experimental errors accompanied have become serious obstacles in the experiments.

Moreover, it has been also known that the aerosol which develops when sample loading buffer is added to the PCR product frequently induces carry-overcontamination and leads to a false positive response, which has been an important problem to be solved, especially when used in diagnosis of diseases.

U.S. Pat. No. 6,153,412 discloses a lyophilized and room temperature stable polymerase chain reaction reagent which is prepared by lyophilizing an aqueous reaction mixture comprising a reaction buffer, MgCl₂, dNTPs, a DNA polymerase, a stabilizing and sedimenting agent selected from the group consisting of FICOLL and sucrose, a water soluble dye selected from the group consisting of bromophenol blue, xylene cyanol, bromocresol red, and cresol red, and a primer. However, the performance of PCR of the lyophilized PCR mixture is declined compared to non-lyophilized mixture. Although the patent disclose that the lyophilized PCR mixture was stable at 55° C. until 136 hours, this application shows that the lyophilized PCR mixture only stable at 55° C. for 1 day (Example 1). The lyophilized PCR mixture is not stable at a high temperature of 55° C. Thus, there is still a need to provide a PCR reaction which is mixture stable at high temperature without performance decline.

### SUMMARY OF THE INVENTION

The invention provides a kit for Polymerase Chain Reaction (PCR), comprising a vessel and at least two set of reagents disposed separately in the vessel, wherein each of the at least two set of reagents comprise at least one component selected from the group consisting of a DNA polymerase, nucleoside triphosphates, reaction buffer, and salt, and provided that each set of the at least two set of reagents is different from each other and combination of each of the at least two set of reagents comprises a DNA polymerase, nucleoside triphosphates, reaction buffer, and salt.

The present invention also provides a kit for Polymerase Chain Reaction (PCR), comprising a vessel and a set of reagents disposed in the vessel, wherein the reagents comprise an agglomerating agent and at least one component selected from the group consisting of a DNA polymerase, nucleoside triphosphates, reaction buffer, and salt.

### BRIEF DESCRIPTION OF THE DRAWINGS

FIG. 1 illustrates the configurations of reagent in microcapsule. A set of reagents in at least two microcapsules (top), at least two set of reagents in one microcapsule (middle), and at least two set of reagents in encapsuled microcapsule (bottom).

FIG. 2 illustrates the electrophoresis pattern PCR products amplified with lyophilized PCR mixture after incubation at 4° C. or 55° C. for certain period of time.

FIG. 3 illustrates the electrophoresis pattern PCR products amplified with dried PCR mixture after incubation at 4° C. or 55° C. for 24 hours.

FIG. 4 illustrates the electrophoresis pattern PCR products amplified with dried PCR mixture as one mixed dot (upper panel) or two separated dots (lower panel) after incubation at 4° C., 25° C. or 55° C. for 24 hours.

FIG. 5 illustrates the electrophoresis pattern PCR products amplified with dried PCR mixture as one mixed dot (upper panel) or three separated dots (lower panel) after incubation at 4° C., 25° C. or 55° C. for 24 hours.

FIG. 6 illustrates the electrophoresis pattern PCR products amplified with dried PCR mixture as one mixed dot (upper panel) or four separated dots (lower panel) after incubation at 4° C., 25° C. or 55° C. for 24 hours.

FIG. 7 illustrates the electrophoresis pattern PCR products amplified with dried PCR mixture containing ammonium sulfate as one mixed dot (lane 2-4) or four separated dots (lane 5-7) after incubation at 4° C., 25° C. or 55° C. for 24 hours.

FIG. 8A illustrates the appearance of PCR mixture with low melting point agarose. 0.5%, 0.3%, 0.15% or 0.08% concentrations of low melting point agarose was added to two-fold concentrated premixed PCR reagent, respectively. After the reaction reagent is solidified, the reaction tube is inverted and the degree of solidification is measured by applying an external force.

FIG. 8B illustrates the appearance of dilution of gel-form PCR mixture. PCR mixture with different concentrations of low melting point agarose were twice diluted by adding DNA template, primer, and water to the reaction tube. The concentrations of low melting point agarose were 0.5%, 0.3%, 0.15% or 0.08%, respectively.

FIG. 8C illustrates the electrophoresis pattern PCR products amplified with gel form PCR mixture with different concentrations of low melting point agarose. The concentrations of low melting point agarose were 0.5%, 0.3%, 0.15% or 0.08%, respectively.

FIG. 9 illustrates the electrophoresis pattern PCR products amplified with two-fold concentrated PCR mixture after stored at 37° C. or at room temperature for 10 days. From left to right, PCR products amplified with two-fold concentrated PCR mixture (2× Mix) after stored at 37° C., with two-fold concentrated PCR mixture with 0.3% low melting point agarose (2×0.3% LM Mix) after stored at 37° C., and with two-fold concentrated PCR mixture (2× Mix) after stored at room temperature.

### DETAILED DESCRIPTION OF THE INVENTION

The present invention provides a kit for Polymerase Chain Reaction (PCR), comprising a vessel and at least two set of reagents disposed separately in the vessel, wherein each of the at least two set of reagents comprise at least one component selected from the group consisting of a DNA polymerase, nucleoside triphosphates, reaction buffer, and salt, and provided that each set of the at least two set of reagents is different from each other and combination of each of the at least two set of reagents comprises a DNA polymerase, nucleoside triphosphates, reaction buffer, and salt required for performing PCR.

The kit for PCR is put to practical use together with other combinations of components depending on the purpose of the use: e.g., distilled water, primers and a template DNA; distilled water and a template DNA; or, distilled water only. For example, for the diagnosis of HIV, HBV and TB (tuberculosis), the PCR reagent may be mixed with their genomic DNA/RNA and complementary primers; and, for DNA sequencing, it may be mixed with the universal or appropriate primers.

In a preferred embodiment, the DNA polymerase is Taq DNA polymerase, KlenTaq DNA polymerase, Pfu DNA polymerase, KOD DNA polymerase, or Tks DNA polymerase.

In a preferred embodiment, the reaction buffer comprises Tris buffer, Tricine buffer, HEPES buffer, and/or combination thereof.

In a preferred embodiment, the nucleoside triphosphates are selected from the group consisting of NTPs, NTP analogs, modified NTPs, dNTPs, dNTP analogs, modified dNTPs, ddNTPs, ddNTP analogs, and modified ddNTPs. In another preferred embodiment, the modified NTPs, dNTPs, or ddNTPs are LNA (locked nucleic acid)-modified NTPs, dNTPs, or ddNTPs.

In a preferred embodiment, the salt is chosen from monovalent salt selected from the group consisting of NH₄⁺, Li⁺, Na⁺, K⁺, Rb⁺, Cs⁺, Ag⁺ and Co⁺ salts; divalent cation selected from the group consisting of Ca²⁺, Mg²⁺, Mn²⁺, Zn²⁺, Cu²⁺, Cd²⁺, Fe²⁺, Sr²⁺ and Co²⁺ salts; trivalent cation selected from the group consisting of Al³⁺, Cr³⁺, Fe³⁺ and Co³⁺ salts. In another preferred embodiment, the salt comprises Mg²⁺ salt, Mn²⁺ salt, K⁺ salt, Na⁺ salt, and/or combination thereof.

In a preferred embodiment, the Mg²⁺ salt comprise Mg(OAc)₂, MgCl₂, MgSO₄, and/or combination thereof.

In a preferred embodiment, the at least two set of reagents further comprise at least one other component selected from the group consisting of a dye, and a PCR stabilizer.

In a preferred embodiment, the dye comprises bromophenol blue, xylene cyanol, cresol red, Orange G, and/or combination thereof.

In a preferred embodiment, the PCR stabilizer comprises DTT, BSA, gelatin, Betaine, methionine, Triton X-100, Tween 20, monosaccharide, disaccharide, polysaccharide, polyethylene glycol, and/or combination thereof.

In a preferred embodiment, the kit is stable at a temperature from −20° C. to 55° C. Preferably, the kit is stable at room temperature.

In a preferred embodiment, the at least two set of reagents is dried or lyophilized. In another preferred embodiment, the at least two set of reagents is in a gel-like form. In further preferred embodiment, the at least two set of reagents is in beads form. In still further preferred embodiment, the at least two set of reagents is in a microcapsule form. In still further preferred embodiment, the at least two set of reagents is independently deposited by combination of the above embodiment. The deposition of the at least two set of reagents is independently selected from drying, lyophilization, gel-like form, beads form, or microcapsule form.

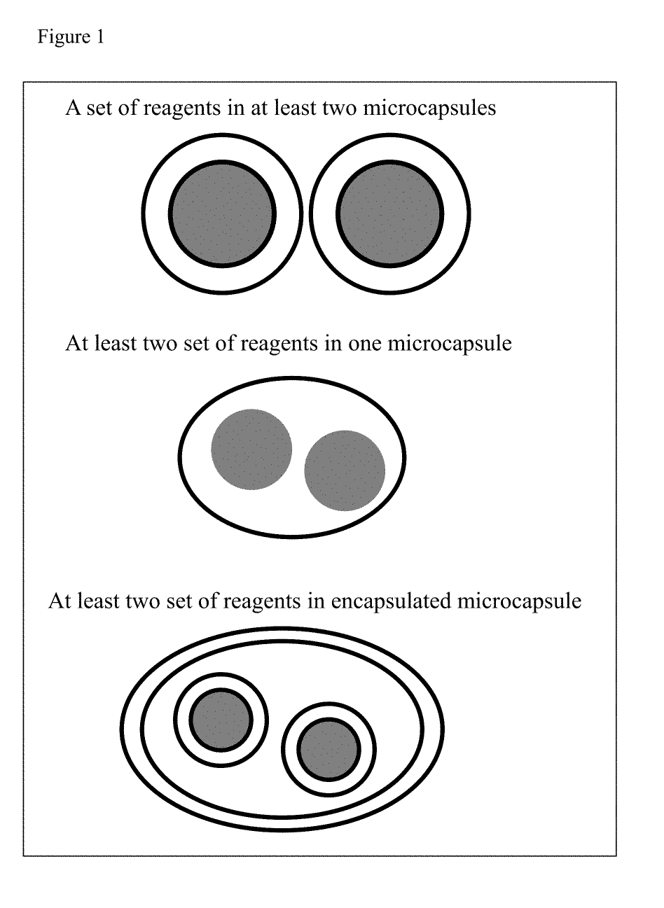

In a preferred embodiment, the microcapsule form comprises a set of reagents in at least two microcapsule (mononuclear or one-core), or at least two set of reagents in one microcapsule (multinuclear or multi-core), or in encapsulated microcapsule (FIG. 1). The microcapsule is selected from the materials with melting point greater than 70° C. The kit or method of the present invention allows the reagents to be grouped. The grouping in different forms allows the reagents gradually released in the reaction process and avoids the interference of reactions.

The present invention also provides a kit for Polymerase Chain Reaction (PCR), comprising a vessel and at least one set of reagents disposed in the vessel, wherein the set of reagents comprise an agglomerating agent and at least one component selected from the group consisting of a DNA polymerase, nucleoside triphosphates, reaction buffer, and salt required for performing PCR.

The kit for PCR is put to practical use together with other combinations of components depending on the purpose of the use: e.g., distilled water, primers and a template DNA; distilled water and a template DNA; or, distilled water only. For example, for the diagnosis of HIV, HBV and TB (tuberculosis), the PCR reagent may be mixed with their genomic DNA/RNA and complementary primers; and, for DNA sequencing, it may be mixed with the universal or appropriate primers.

In a preferred embodiment, the agglomerating agent comprises agarose, gelatin, latex, silica, hydrogels, PAA (poly acrylic acid), PVA (poly vinyl alcohol), Chitosan, PNIPAM (Poly-N-isopropyl acrylamide), substituted PNIPAM (including PNIPAM-aa (poly-N-isopropyl acrylamide-acrylic acid), PNIPAM-allylamine (Poly-N-isopropyl acrylamide-allylamine), and PNIPAM-SH), PAMAM (Polyamidoamine), PEG (Poly ethylene glycol), alginic acid, HPC (hydroxyl propyl cellulose), and/or combination thereof.

In a preferred embodiment, the set of reagents is in one to ten-fold concentrated. In still further preferred embodiment, the set of reagents is in two to five-fold concentrated.

In a preferred embodiment, the DNA polymerase is Taq DNA polymerase, KlenTaq DNA polymerase, Pfu DNA polymerase, KOD DNA polymerase, or Tks DNA polymerase.

In a preferred embodiment, the reaction buffer comprises Tris buffer, Tricine buffer, HEPES buffer, and/or combination thereof.

In a preferred embodiment, the nucleoside triphosphates are selected from the group consisting of NTPs, NTP analogs, modified NTPs, dNTPs, dNTP analogs, modified dNTPs, ddNTPs, ddNTP analogs, and modified ddNTPs. In still further preferred embodiment, the modified NTPs, dNTPs, or ddNTPs are LNA (locked nucleic acid)-modified NTPs, dNTPs, or ddNTPs.

In a preferred embodiment, the salt is chosen from monovalent salt selected from the group consisting of NH₄⁺, Li⁺, Na⁺, K⁺, Rb⁺, Cs⁺, Ag⁺ and Co⁺ salts; divalent cation selected from the group consisting of Ca²⁺, Mg²⁺, Mn²⁺, Zn²⁺, Cu²⁺, Cd²⁺, Fe²⁺, Sr²⁺ and Co²⁺ salts; trivalent cation selected from the group consisting of Al³⁺, Cr³⁺, Fe³⁺ and Co³⁺ salts. In still further preferred embodiment, the salt comprises Mg²⁺ salt, Mn²⁺ salt, K⁺ salt, Na⁺ salt, and/or combination thereof.

In a preferred embodiment, the Mg²⁺ salt comprise Mg(OAc)₂, MgCl₂, MgSO₄, and/or combination thereof.

In a preferred embodiment, the set of reagents further comprise at least one other component selected from the group consisting of a dye, and a PCR stabilizer.

In a preferred embodiment, the dye comprises bromophenol blue, xylene cyanol, cresol red, Orange G, and/or combination thereof.

In a preferred embodiment, the PCR stabilizer comprises DTT, BSA, gelatin, Betaine, methionine, Triton X-100, Tween 20, monosaccharide, disaccharide, polysaccharide, polyethylene glycol, and/or combination thereof.

In a preferred embodiment, the kit is stable at a temperature from −20° C. to 55° C. In still further preferred embodiment, the kit is stable at a temperature from room temperature to 37° C.

It has been well known that: materials such as gelatin, bovine serum albumin (BSA), ammonium sulfate or Thesit etc., stabilize a DNA polymerase and dNTPs, and non-ionic surfactants such as NP40 and Tween 20 etc., improve the reactivity of the PCR mixture. The inventors, however, determined that ammonium sulfate may have an effect on the level of PCR process seriously when it is employed in lyophilized PCR reagent. Accordingly, PCR reagent of the present invention preferably comprises a stabilizer such as gelatin, BSA, Thesit, PEG-8000 (polyethyleneglycol-8000) or polyol (e.g., ficoll, sucrose, glycerol, glucose, mannitol, galacitol, glucitol and sorbitol), most preferably, polyol, since polyol is determined to play a role as a sedimenting agent.

The kit of the present invention may further comprise a sedimenting agent or a water-soluble dye in the presence/absence of stabilizer. The polyol plays dual role as a stabilizer and a sedimenting agent, and most preferably, glucitol, glucose, ficoll and sucrose which are kinds of polyol, may be added to the reagent. As a water-soluble dye, Orange G, bromophenol blue, xylene cyanole, bromocresol red, cresol red, etc., may be added to the reagent. The water-soluble dye facilitates to identify complete mixing of the PCR reagent and test sample, and saves experimenters the trouble of adding a sample loading buffer which is required for analysis of PCR product, thereby preventing the carry-over contamination.

The kit of the present invention provides advantages as follows: it simplifies the multi-step PCR manipulation in which every component of the reaction mixture for PCR is to be added to each of test samples; it increases the heat stability of the reaction mixture; it prevents carry-over contamination by skipping the step of adding the sample loading buffer which is necessary for analysis of PCR products; it improves credibility for PCR in diagnosis of diseases and in performance of repeated experiments, by excusing any possibility of committing experimental errors caused by mispipetting; it can be stored at room temperature until 6 months.

The kit of the present invention is developed as a kit for analysis of nucleotide sequence or diagnosis of diseases, and for DNA amplification of a specific region of genome as well.

### EXAMPLES

The examples below are non-limiting and are merely representative of various aspects and features of the present invention.

### Example 1: Determination of Heat Stability of Lyophilized PCR Mixture

PCR mixture containing Taq DNA polymerase, Tris-HCl, KCl, MgCl₂, Triton X-100, BSA, dNTPs, Orange G, sucrose, and sorbitol was lyophilized and further incubated at 4° C. or 55° C. to determine heat stability of lyophilized PCR mixture.

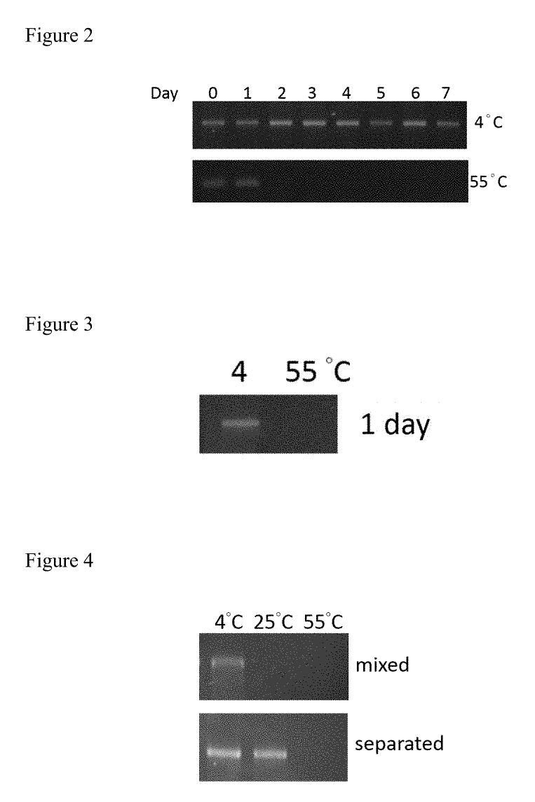

PCR amplification was carried out with reagent as above. A pair of specific primers (5′-GAGCGGATAACAATTTCACACAG-3′ and 5′-GGGTTATTGTCTCATGAGCG-3′) was used to amplify a 665 bp amplicon from 1 pg pUC19 plasmid as template. PCR was preform for 35 cycles with parameters as follows: denature at 95° C. for 20 sec, anneal at 55° C. for 15 sec and extend at 72° C. for 15 sec. PCR was performed every 24 hr in the same manner, and the resultant PCR products were electrophoresed (FIG. 2). As shown in FIG. 2, lyophilized PCR mixture was stable for at least 7 days at 4° C. However, lyophilized PCR mixture was stable for 1 day at 55° C.

### Example 2: Determination of Heat Stability of Dried PCR Mixture

PCR mixture containing Taq DNA polymerase, Tris-HCl, KCl, MgCl₂, Triton X-100, BSA, dNTPs, Orange G, and PEG-8000 was dried in 55° C. for 20 min and further incubated at 4° C. or 55° C. to determine heat stability of dried PCR mixture. PCR was performed after 24 hr incubation in the same manner as Example 1, and the resultant PCR products were electrophoresed (FIG. 3). As shown in FIG. 3, dried PCR mixture was not stable at 55° C.

### Example 3: Determination of Heat Stability of Dried PCR Components Separated in Two Mixture Sets

PCR mixture was separated into two sets, one set contained Tris-HCl, Triton X-100, BSA, dNTP, Taq DNA polymerase and PEG-8000, and the other set contained Tris-HCl, KCl, MgCl₂, Orange G and PEG-8000. The two sets of PCR reagents were pre-loaded into same PCR tube as two separated dots or one mixed dot and further dried in 55° C. for 20 min followed by incubation at 4° C., 25° C. or 55° C. to determine heat stability of two sets of dried PCR mixture. PCR was performed after 24 hr incubation in the same manner as Example 1, and the resultant PCR products were electrophoresed (FIG. 4). As shown in FIG. 4, dried PCR mixture as one mixed dots was not stable at temperature more than 25° C. However, dried PCR mixture as two separated dots was stable at room temperature for at least 24 hours.

### Example 4: Determination of Heat Stability of Dried PCR Components Separated in Three Mixture Sets

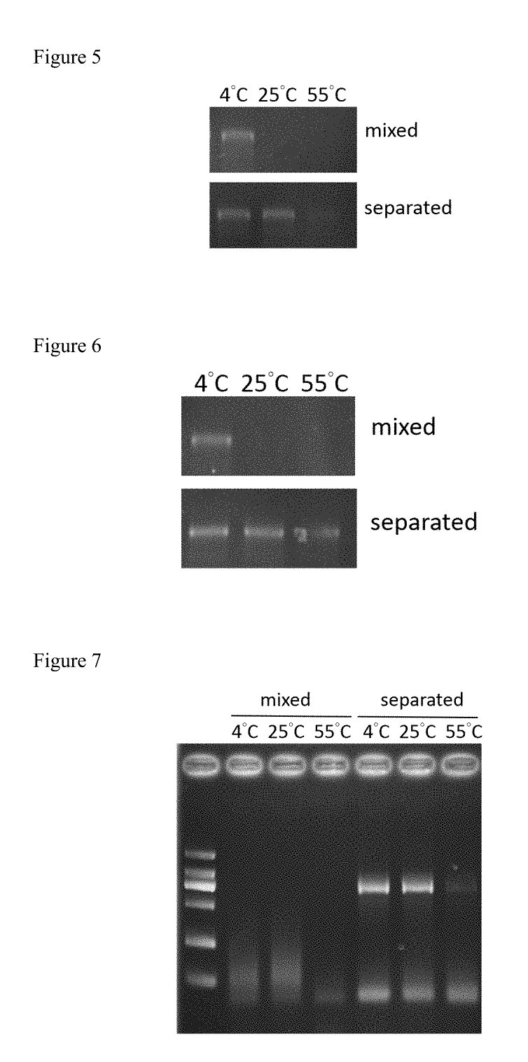

PCR mixture was separated into three sets, the first set contained Tris-HCl, Triton X-100, BSA, Taq DNA polymerase and PEG-8000, the second set contained dNTP and PEG-8000, and the third set contained Tris-HCl, KCl, MgCl₂, Orange G and PEG-8000. The three sets of PCR reagents were pre-loaded into same PCR tube as three separated dots or one mixed dot and further dried in 55° C. for 20 min followed by incubation at 4° C., 25° C. or 55° C. to determine heat stability of three sets of dried PCR mixture. PCR was performed after 24 hr incubation in the same manner as Example 1, and the resultant PCR products were electrophoresed (FIG. 5). As shown in FIG. 5, dried PCR mixture as three separated dots was stable at 25° C. and maintained few PCR activity at 55° C. for at least 24 hours.

### Example 5: Determination of Heat Stability of Dried PCR Components Separated in Four Mixture Sets

PCR mixture was separated into four sets, the first set contained Tris-HCl, Triton X-100, BSA, Taq DNA polymerase and PEG-8000, the second set contained dNTP and PEG-8000, and the third set contained Tris-HCl, KCl, MgCl₂ and PEG-8000, and the fourth set contained Orange G and PEG-8000. The four sets of PCR reagents were pre-loaded into same PCR tube as four separated dots or one mixed dot and further dried in 55° C. for 20 min followed by incubation at 4° C., 25° C. or 55° C. to determine heat stability of four sets of dried PCR mixture. PCR was performed after 24 hr incubation in the same manner as Example 1, and the resultant PCR products were electrophoresed (FIG. 6). As shown in FIG. 6, dried PCR mixture as four separated dots was stable at 25° C. and 55° C. for at least 24 hours.

### Example 6: Determination of Heat Stability of Dried PCR Components Containing Ammonium Sulfate and Separated in Four Mixture Sets

Ammonium sulfate played an enhancer role in PCR reaction, however, it reduced heat stability of PCR mixture.

PCR mixture was separated into four sets, the first set contained Tris-HCl, Triton X-100, BSA, Taq DNA polymerase and PEG-8000, the second set contained dNTP and PEG-8000, and the third set contained Tris-HCl, KCl, MgCl₂ and PEG-8000, and the fourth set contained Orange G, ammonium sulfate and PEG-8000. The four sets of PCR reagents were pre-loaded into same PCR tube as four separated dots or one mixed dot and further dried in 55° C. for 20 min followed by incubation at 4° C., 25° C. or 55° C. to determine heat stability of four sets of dried PCR mixture. PCR was performed after 24 hr incubation in the same manner as Example 1, and the resultant PCR products were electrophoresed (FIG. 7). As shown in FIG. 7, dried PCR mixture as four separated dots was stable at 25° C. for at least 24 hours, while PCR mixture as one mixed dot was unstable even store at 4° C.

### Example 7: Degree of Solidification, Dissolvability, and the Effect on PCR Products Amplification of Adding Different Concentrations of Agglomerating Agent to Premixed PCR Reagent

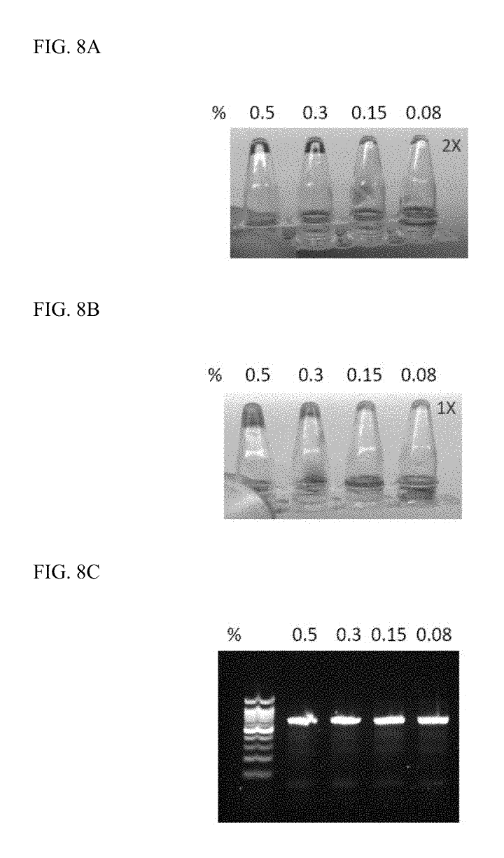

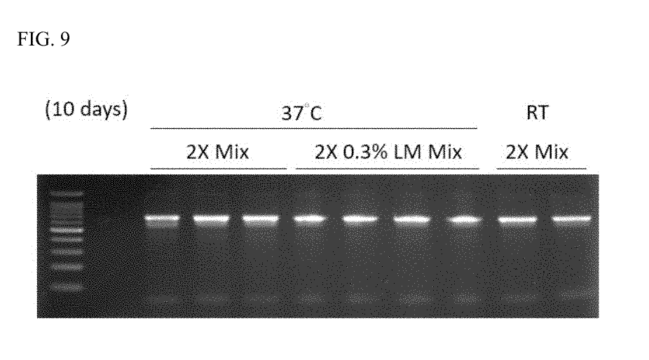

Low melting point agarose was chosen as agglomerating agent to test the degree of solidification, dissolvability, and the effect on PCR products amplified of adding agglomerating agent to premixed PCR reagents. As shown in FIG. 8A, 0.5%, 0.3%, 0.15% or 0.08% low melting point agarose were added to two-fold concentrated PCR premixed reagents, respectively, and the degree of solidification with different concentrations of agglomerating agent was measured by applying external force. After applying external force, premixed PCR reagents with 0.5% or 0.3% low melting point agarose remained solidified, while premixed PCR reagents with 0.15% or 0.08% low melting point agarose were partially liquefied.

To test the dissolvability of gel-form premixed PCR reagents, PCR mixture with 0.5%, 0.3%, 0.15% or 0.08% concentrations of low melting point agarose were twice diluted by adding DNA template, primer, and water to the reaction tube, respectively (FIG. 8B). PCR mixture with 0.3%, 0.15% or 0.08% concentrations of low melting point agarose were partially to totally dissolved, while PCR mixture with 0.5% concentrations of low melting point agarose remained solidified.

The effect of adding low melting point agarose to PCR reagents on PCR products amplified were examined by gel electrophoresis (FIG. 8C). Electrophoresis pattern of PCR products showed that the different dosage of low melting point agarose did not affect the polymerase chain reaction efficacy.

### Example 8: Efficacy of PCR Products Amplification with Premixed PCR Reagents with or without Agglomerating Agent at Different Storage Conditions

To test the efficacy of PCR products amplification with premixed PCR reagents with or without agglomerating agent under long period of different storage temperature, PCR products amplified with two-fold concentrated PCR mixture after stored at 37° C., with two-fold concentrated PCR mixture with 0.3% low melting point agarose after stored at 37 degrees Celsius, and with two-fold concentrated PCR mixture after stored at room temperature were examined by gel electrophoresis. The result showed that the gel-form concentrated pre-mixed PCR reagents did not affect the polymerase chain reaction efficacy (FIG. 9).

What is claimed is:

1. A kit for Polymerase Chain Reaction (PCR), comprising a vessel and at least two set of reagents disposed separately in the vessel, wherein each of the at least two set of reagents comprise at least one component selected from the group consisting of a DNA polymerase, nucleoside triphosphates, reaction buffer, and salt, and provided that each set of the at least two set of reagents is different from each other and combination of each of the at least two set of reagents comprises a DNA polymerase, nucleoside triphosphates, reaction buffer, and salt.

2. The kit of claim 1, wherein the DNA polymerase is Taq DNA polymerase, KlenTaq DNA polymerase, Pfu DNA polymerase, KOD DNA polymerase, or Tks DNA polymerase.

3. The kit of claim 1, wherein the reaction buffer comprises Tris buffer, Tricine buffer, HEPES buffer, and/or combination thereof.

4. The kit of claim 1, wherein the nucleoside triphosphates are selected from the group consisting of NTPs, NTP analogs, modified NTPs, dNTPs, dNTP analogs, modified dNTPs, ddNTPs, ddNTP analogs, and modified ddNTPs.

5. The kit of claim 1, wherein the salt comprises Mg²⁺ salt, Mn²⁺ salt, K⁺ salt, Na⁺ salt, and/or combination thereof.

6. The kit of claim 1, wherein the at least two set of reagents further comprise at least one other component selected from the group consisting of a dye, and a PCR stabilizer.

7. The kit of claim 6, wherein the dye comprises bromophenol blue, xylene cyanol, cresol red, Orange G, and/or combination thereof.

8. The kit of claim 6, wherein the PCR stabilizer comprises DTT, BSA, gelatin, Betaine, methionine, Triton X-100, Tween 20, monosaccharide, disaccharide, polysaccharide, polyethylene glycol, and/or combination thereof.

9. The kit of claim 1, which is stable at a temperature from −20° C. to 55° C.

10. A kit for Polymerase Chain Reaction (PCR), comprising a vessel and at least one set of reagents disposed in the vessel, wherein the set of reagents comprise an agglomerating agent and at least one component selected from the group consisting of a DNA polymerase, nucleoside triphosphates, reaction buffer, and salt.

11. The kit of claim 10, wherein the agglomerating agent comprises agarose, gelatin, latex, silica, hydrogels, PAA (poly acrylic acid), PVA (poly vinyl alcohol), Chitosan, PNIPAM (Poly-N-isopropyl acrylamide), substituted PNIPAM (including PNIPAM-aa (poly-N-isopropyl acrylamide-acrylic acid), PNIPAM-allylamine (Poly-N-isopropyl acrylamide-allylamine), and PNIPAM-SH), PAMAM (Polyamidoamine), PEG (Poly ethylene glycol), alginic acid, HPC (hydroxyl propyl cellulose), and/or combination thereof.

12. The kit of claim 10, wherein the set of reagents is in one to ten-fold concentrated.

13. The kit of claim 10, wherein the DNA polymerase is Taq DNA polymerase, KlenTaq DNA polymerase, Pfu DNA polymerase, KOD DNA polymerase, or Tks DNA polymerase.

14. The kit of claim 10, wherein the reaction buffer comprises Tris buffer, Tricine buffer, HEPES buffer, and/or combination thereof.

15. The kit of claim 10, wherein the nucleoside triphosphates are selected from the group consisting of NTPs, NTP analogs, modified NTPs, dNTPs, dNTP analogs, modified dNTPs, ddNTPs, ddNTP analogs, and modified ddNTPs.

16. The kit of claim 10, wherein the salt comprises Mg²⁺ salt, Mn²⁺ salt, K⁺ salt, Na⁺ salt, and/or combination thereof.

17. The kit of claim 10, wherein the set of reagents further comprise at least one other component selected from the group consisting of a dye, and a PCR stabilizer.

18. The kit of claim 17, wherein the dye comprises bromophenol blue, xylene cyanol, cresol red, Orange G, and/or combination thereof.

19. The kit of claim 17, wherein the PCR stabilizer comprises DTT, BSA, gelatin, Betaine, methionine, Triton X-100, Tween 20, monosaccharide, disaccharide, polysaccharide, polyethylene glycol, and/or combination thereof.

20. The kit of claim 10, which is stable at a temperature from −20° C. to 55° C.
